(19) United States
(12) Patent Application Publication
(10) Pub. No.: US 2016/0367374 A1
(43) Pub. Date: Dec. 22, 2016

(54) IMPLANTS

(71) Applicant: CERAMTEC GMBH, Plochingen (DE)

(72) Inventors: Heinrich WECKER, Eckental (DE); Alfons KELNBERGER, Röthenbach (DE); Frank ZIERMANN, Berlin (DE)

(73) Assignee: CERAMTEC GMBH, Plochingen (DE)

(21) Appl. No.: 15/101,860

(22) PCT Filed: Dec. 5, 2014
(86) PCT No.: PCT/EP2014/003256

§ 371 Date: Jun. 3, 2016

(30) Foreign Application Priority Data
Dec. 5, 2013 (DE) 10 2013 224 994.2

(51) Int. Cl. A61F 2/40; A61L 27/56; A61L 27/34; A61L 27/30; A61L 27/52; A61L 27/12; A61L 27/10; A61L 27/50

(52) U.S. Cl. CPC A61F 2/4014; A61L 27/10; A61L 27/56; A61L 27/50; A61L 27/306; A61L 27/52; A61L 27/12; A61L 27/34; A61F 2/4003; A61L 2400/18; A61L 2430/24; A61F 2210/0076

(57) ABSTRACT

The present invention relates to implants, methods for the production thereof and the use thereof.

The present invention relates to implants, methods for production thereof and use thereof.

Implants for partial restoration of joint surfaces are known and are implanted when irreparable cartilage damage occurs, for example due to arthritis, in the most varied joints in the human body and leads to impairments. If for example joint surfaces are damaged or destroyed by wear, disease or injuries, and neither conservative non-surgical treatment methods nor joint-preserving surgery promises successful healing, the implantation of a partial prosthesis is generally necessary. These partial endoprosthetic implants may be understood as a treatment concept in patients for whom total endoprosthetic surgery would be too risky or would not be suitable because of their active lifestyle. In this way, in order to increase mobility and quality of life in everyday life, at work and at leisure, on the one hand the pain in the affected joint is eliminated or reduced in the long term and on the other hand the maximum possible mobility of this joint is restored.

In contrast to total endoprostheses, in this case only a part of the articulation surface is restored. As a rule the surgery can be performed in a minimally invasive manner.

Nevertheless, the potential remains for a revision for a total endoprosthesis. This can take place at a later time. Older patients could likewise benefit from this if, for example, they refuse a total joint replacement because of the higher morbidity.

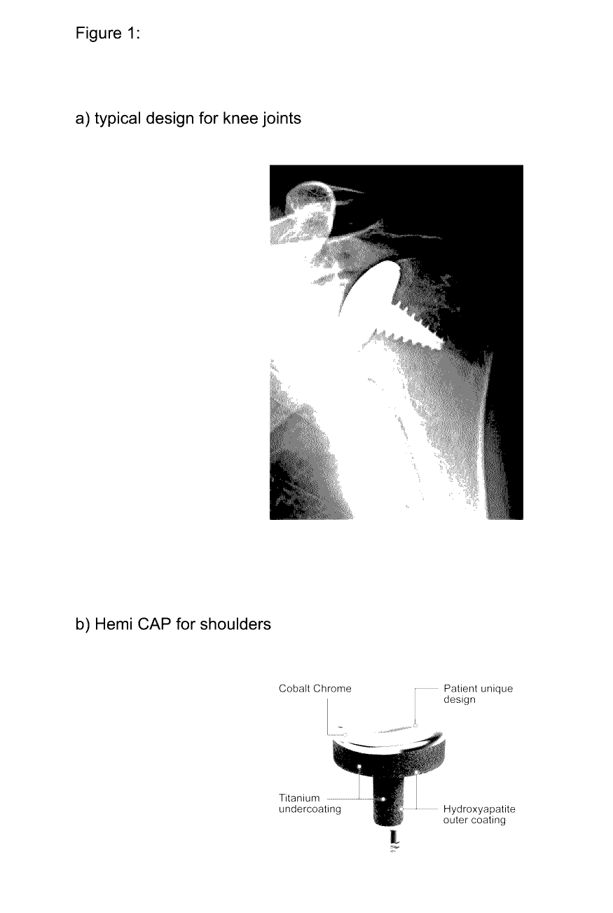

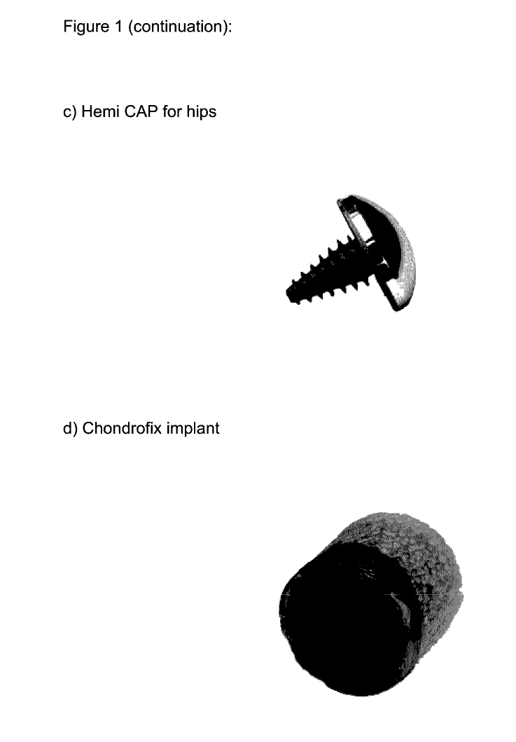

Such prosthetic components for hip, knee, shoulder and small joints, based on metal materials such as titanium or cobalt chromium, are commercially available, wherein these implants function in two parts. Examples of such components are shown in FIG. 1.

As a rule, they have a tribologically stressed part which serves as an articulation surface and an osseointegrating part which grows into the bone tissue and ensures secure anchoring.

The disadvantages occurring with the metallic solutions are known:
metal abrasion and negative effects on the human organism resulting therefrom,
artefacts in imaging for medical diagnostics,
ageing effects and long-term behavior (fatigue, corrosion, release of metal ions which may have a toxic effect)

An increasing risk of infection during the operation occurs more and more frequently as a general problem.

Moreover, in addition to these metallic solutions a so-called osteochondral implant is known, which is based on human cartilage and bone tissue and can be implanted in a minimally invasive manner in the damaged regions (FIG. 1d). In such implants the high wear, the low strengths, ageing effects and insufficient long-term behavior have disadvantageous effects.

The object of the present invention is to provide implants which do not have the disadvantages of the known solutions.

The implants according to the invention should in particular have the following characteristics:
After the implantation, the implant according to the invention should grow together reliably with the bone tissue of the joint and should ensure sufficiently strong anchoring and stability.
After the implantation, the implant according to the invention together with the rest of the joint surface should constitute a compact unit from the tribological point of view and should be operatively connected securely and permanently to the articulation partner.
In articulation against a natural cartilage surface, the implant according to the invention should function reliably, durably and with low wear.
The implant according to the invention should ideally be configured so that it favors and promotes the formation of new, natural cartilage tissue.
The implant according to the invention should not cause any harm to health during its residence time in the human body. In particular:
   it should not produce any abrasive particles which are harmful to health
   in interaction with the articulation partner, in particular with the natural cartilage tissue, it should not cause long-term damage to the articulation partner
   it should not be damaged or destroyed by the biomechanical conditions
   it should not offer favorable conditions for bacteria which cause infections.

The object on which the invention is based is achieved by implants having the features of the main claim. Preferred embodiments are set out in the subordinate claims.

The invention relates to ceramic/polymer composite-based implants.

In principle the implant according to the invention consists of three different functional units (hereafter also referred to as layers):
The first unit is an osseointegrating ceramic-based unit, which forms a biologically active connection with the bone tissue lying below the cartilage.
The second unit is likewise a ceramic-based unit, which is connected to the first and serves primarily as substrate for the third unit. This can also be tribologically stressed in the event of wear of the third unit and can serve as an articulation surface.
The third unit is a polymer-based unit, preferably based on PVA or hydrogels, which is firmly connected to the second unit and primarily ensures the tribological function in the articulation with the natural cartilage. A hydrogel is a polymer which contains water but is water-insoluble, and of which the molecules are combined into a three-dimensional network chemically, for example by covalent or ionic bonds, or physical, for example by entangling the polymer chains.

The osseointegrative first unit has a ceramic with porous proportions and open-pored, interconnecting structure. It can be produced in a manner which is known per se, for example by means of
molding processes
freeze direct foam process
foaming processes
pore-forming methods based on organic pore generators

These structures should be optimally adapted to the biological processes of osteogenesis and vascularization and should ensure optimal osseoconductive characteristics.

Typical parameters of such a structure are pore diameters of the order of magnitude between 100 and 1000 μm, especially between 300 and 700 μm.

Open porosities are of the order of magnitude between 50% and 90%, preferably between 60% and 80%. The moduli of elasticity should be of the order of magnitude between 5 and 50 GPa, ideally in the range of human bone, in order to ensure an advantageous mechanical stimulus for the formation of bone substance.

In addition these structures can be coated with osseoinductive coatings of any type in a manner which is known per se, so that the effect of the osseointegration is further enhanced.

Examples of such coatings which can be used according to the invention are
bioglasses, especially the composition 45S5
hydroxylapatite coatings of any type, including nanostructured and biomimetically acting layers
phosphating layers, in particular covalently bonded, monolayer phosphating layers
metallic coatings, in particular based upon tantalum or titanium

The second unit has a relatively dense ceramic which is characterized in particular by its substantial hardness and strength. It is firmly connected to the first unit and on the side facing the third unit is structured so that it constitutes an optimal substrate surface for the third unit.

The connection to the first unit can be made for example by means of application of a slip in the green state and subsequent co-sintering, or according to a method for producing the first unit in one piece.

The structure of the side facing the third, polymer-based unit is particularly important. This side must not only ensure a firm positive or non-positive connection, but also a dispersion during the biomechanical loading of the third unit or the lowest possible shearing loads. In particular the occurrence of internal cracks or other damage of the cross-linked polymer should be avoided.

Specifically, in the structuring it must ensured that no sharp edges are produced.

Quite generally, according to the invention structures are provided which increase the surface and thus ensure a good connection between the second unit and the third unit, for example by means of adhesion forces or also by means of medically suitable glues. Suitable possibilities in this connection are for example defined indentations in the surface, which may have the appearance of a golf ball structure, or also undercut or drop-shaped indentations in the range from several μm to mm, which offer optimal support to the polymer materials of the third unit and lead to a self-stabilization or self-fixing under axial loading, or also web-like structures.

It is particularly advantageous if, under load, the strength of the connection between the second and third units is increased. According to the invention this is achieved by appropriately configured structures on the ceramic second unit.

Ideally the structure of the second unit, which faces the third unit, has a very slight microroughness in the region of a few μm, which can be achieved by regrinding or polishing, or at least those regions of the structure which are exposed in the event of any abrasion of the third unit.

Thus, even in the event of destruction or abrasion of the third unit, an optimal articulation of the ceramic surface of the second unit with the cartilage can be ensured.

The structuring of the ceramic of the second unit can take place by means of injection molding processes (ceramic injection molding, low pressure injection molding) or other molding technologies, also by mechanical processing in the green state or by laser processing or ultrasonically assisted mechanical processing in the sintered state, or also spark erosion or chemical etching processes.

The ceramic material of the first and the second unit is preferably an oxide ceramic from the class of aluminum oxides or zirconium oxides, for example zirconium-reinforced aluminum oxide or yttrium-stabilized zirconium oxide, but also all variants thereof or composite materials with the general designations ZTA (zirconia-toughened alumina) or ATZ (alumina-toughened zirconia).

Non-oxide ceramics, such as for example materials based on Si₃N₄, are likewise possible according to the invention.

Because of their chemical composition, hardness and strength all the listed materials have extremely good tribological characteristics, and during the implantation they are to a large extent damage-tolerant and are more than capable of meeting the biomechanical requirements.

Moreover, without further functionalization they are to a large extent bioinert and prevent the proliferation of bacteria.

Advantageously, all further developments of these materials are for example extremely damage-tolerant materials such as for example rare earth stabilized dispersoid ceramics consisting of zirconium oxide with fractions of aluminates.

For the third unit three-dimensionally linked polymers have proved to be particularly suitable polymers which, in addition to their mechanical characteristics in particular with respect to rigidity and shearing load capacity, also withstand the biomechanical loads, and which are similar to natural cartilage material.

Moreover, polymers have the advantage that they can be charged with functional groups by which the physical characteristics can be set in a targeted manner.

In particular hydrogels can be used as carriers of biologically active substances which can give rise to antibacterial or chondrogenetic effect. These biologically active substances favor the in vivo formation of endogenous cartilage material.

Often in this connection synthetic alginates are also used in combination with human stem cells, in order thus to promote the formation of endogenous cartilage tissue. This concept could also be integrated into the third unit.

It is very advantageous if the internal structure of the polymers, in particular the hydrogels, is configured so that the cartilage formation is promoted under compressive mechanical load and with corresponding charging with chondrogenetic substances. These mechanisms also act in the natural cartilage which is dependent upon mechanical stimuli.

The fixed connection between second and third units can be produced by positive or non-positive joining processes, but it is also conceivable—especially in the case of undercut structures—to melt the polymers or generally to apply them in the liquid phase.

The thickness provided according to the invention for the polymer or hydrogel layer is highly dependent upon the characteristics of the polymer. Micro- or nanocoatings can be applied or joined to the second unit or also thick layers up to several mm.

Moreover, with respect to the topographical configuration of the articulating surface it may be extremely advantageous if this surface is adapted to the anatomical and patient-specific situation, which can be ensured by means of suitable CAD-CAM methods in the manufacturing process.

Furthermore, a perfect fit of the implant from the biomechanical and surgical viewpoint is extremely advantageous, which can be ensured by a suitable procedure with corresponding and optionally customized instruments.

The implants according to the invention are preferably used for restoration of joint surfaces in the human body, for example joint surfaces from the shoulder, hip, knee and foot regions. The implants according to the invention are suitable as joint surfaces for local cartilage defects, as partial joint replacements but also as full endoprostheses. They are particularly suitable in articulations against natural cartilage surfaces, so-called hemiprostheses, for example as a shoulder hemiprosthesis, also known as a humeral head prosthesis. This involves a partial replacement of the shoulder joint, in which the natural shoulder socket (glenoid) is retained and only the humeral head is replaced by an endoprosthesis.

To summarize, the present invention relates to the following:
Ceramic/polymer composite-based implants.
Implants according to point 1, characterized in that they contain three different functional layers.
Implants according to point 1 or 2, characterized in that the first layer is ceramic-based.
Implants according to one or more of the preceding points, characterized in that the second layer is likewise ceramic-based and is connected to the first layer.
Implants according to one or more of the preceding points, characterized in that the third layer is polymer-based and is firmly connected to the second layer.
Implants according to one or more of the preceding points, characterized in that the first layer has an osseointegrating characteristic.
Implants according to one or more of the preceding points, characterized in that the second layer serves primarily as substrate for the third layer.
Implants according to one or more of the preceding points, characterized in that the third layer primarily ensures the tribological function in the articulation with the natural cartilage.
Implants according to one or more of the preceding points, characterized in that the first layer contains a ceramic with porous proportions.
Implants according to one or more of the preceding points, characterized in that the first layer contains a ceramic with porous proportions and open-pored interconnecting structure.
Implants according to one or more of the preceding points, characterized in that the first layer has pore diameters of the order of magnitude between 100 and 1000 μm, especially between 300 and 700 μm.
Implants according to one or more of the preceding points, characterized in that the first layer has open porosities of the order of magnitude between 50% and 90%, preferably between 60% and 80%.
Implants according to one or more of the preceding points, characterized in that the moduli of elasticity of the first layer are of the order of magnitude between 5 and 50 GPa, preferably in the range of human bone.
Implants according to one or more of the preceding points, characterized in that the first layer is coated with osseoinductive coatings.
Implants according to one or more of the preceding points, characterized in that the first layer is coated with osseoinductive coatings, wherein the layer is selected from bioglasses, hydroxylapatite coatings, phosphating layers and/or metallic coatings.
Implants according to one or more of the preceding points, characterized in that the first layer is coated with osseoinductive coatings, wherein the layer is selected from bioglasses, preferably from the bioglass having the composition 45S5, from hydroxylapatite coatings, preferably from nanostructured and biomimetically acting hydroxylapatite layers, phosphating layers, in particular covalently bonded, monolayer phosphating layers and/or metallic coatings, in particular metallic coatings based upon tantalum or titanium.
Implants according to one or more of the preceding points, characterized in that the second layer has a relatively dense ceramic.
Implants according to one or more of the preceding points, characterized in that the second layer has a high hardness and strength.
Implants according to one or more of the preceding points, characterized in that the second layer has a side which faces the third layer and is structured so that it constitutes an optimal substrate surface for the third unit.
Implants according to one or more of the preceding points, characterized in that the second layer has a side which faces the third layer and is structured so that it constitutes an optimal substrate surface for the third layer, which ensures a firm positive or non-positive connection and a dispersion during the biomechanical loading of the third layer or the lowest possible shearing loads.
Implants according to one or more of the preceding points, characterized in that the second layer has structures which increase the surface.
Implants according to one or more of the preceding points, characterized in that the second layer has structures which increase the surface, preferably defined indentations in the surface, (golf ball structure), undercut or drop-shaped indentations in the range from several μm to mm, or web-like structures.
Implants according to one or more of the preceding points, characterized in that the second layer has a side which faces the third layer and has a very slight microroughness in the region of a few μm.
Implants according to one or more of the preceding points, characterized in that the ceramic material of the first and the second layer contains an oxide ceramic from the class of aluminum oxides or zirconium oxides or a non-oxide ceramic, for example materials based on Si₃N₄.
Implants according to one or more of the preceding points, characterized in that the third layer contains three-dimensionally linked polymers.
Implants according to one or more of the preceding points, characterized in that the third layer contains hydrogels.
Implants according to one or more of the preceding points, characterized in that the third layer contains three-dimensionally linked polymers, wherein the internal structure of the polymers, in particular the internal structure of the hydrogels, is configured so that the cartilage formation is promoted under compressive mechanical load and with corresponding charging with chondrogenetic substances.
Use of the implants according to one or more of the preceding points for restoration of joint surfaces in the human body, for example joint surfaces from the shoulder, hip, knee and foot regions.
Use of the implants according to one or more of the preceding points as joint surfaces for local cartilage defects, as partial joint replacements but also as full endoprostheses.
Use of the implants according to one or more of the preceding points in articulations against natural cartilage surfaces.

1. Ceramic/polymer composite-based implant.

2. Implant according to claim 1, characterized in that it contains three different functional layers.

3. Implant according to claim 2, characterized in that a first functional layer is ceramic-based.

4. Implant according to claim 2, characterized in that a second functional layer is ceramic-based and is connected to the first functional layer.

5. Implant according to claim 2, characterized in that a third functional layer is polymer-based and is firmly connected to the second layer.

6. Implant according to claim 3, characterized in that the first functional layer has an osseointegrating characteristic.

7. Implants according to claim 5, characterized in that the second functional layer serves primarily as substrate for the third functional layer.

8. Implant according to claim 5, characterized in that the third functional layer primarily ensures tribological function in the articulation with the natural cartilage.

9. Implant according to claim 3, characterized in that the first functional layer contains a ceramic with porous proportions.

10. Implant according to claim 3, characterized in that the first functional layer contains a ceramic with porous proportions and open-pored interconnecting structure.

11. Implant according to claim 10, characterized in that the first functional layer has pore diameters of the order of magnitude (a) between 100 and 1000 μm, especially or (b) between 300 and 700 μm.

12. Implant according to claim 3, characterized in that the first functional layer has open porosities of the order of magnitude of (a) between 50% and 90%, preferably or (b) between 60% and 80%.

13. Implant according to claim 3, characterized in that the first functional layer has moduli of elasticity of the order of magnitude between 5 and 50 GPa, preferably in the range of human bone.

14. Implant according to claim 3, characterized in that the first functional layer is coated with osseoinductive coatings.

15. Implant according to claim 3, characterized in that the first functional layer is coated with osseoinductive coatings, wherein the layer comprises at least one from among bioglasses, hydroxylapatite coatings, phosphating layers or metallic coatings.

16. Implant according to claim 3, characterized in that the first functional layer is coated with osseoinductive coatings, wherein the layer is selected from bioglasses, preferably from the bioglass having the composition 45S5, hydroxylapatite coatings, preferably nanostructured and biomimetically acting layers, phosphating layers, in particular covalently bonded, monolayer phosphating layers and/or metallic coatings, in particular metallic coatings based on tantalum or titanium.

17. Implant according to claim 4, characterized in that the second layer has a relatively dense ceramic.

18. Implant according to claim 3, characterized in that second functional layer has a high hardness and strength.

19. Implant according to claim 2, characterized in that a second functional layer has a side which faces a third functional layer and is structured so that it constitutes an optimal substrate surface for the third functional layer.

20. Implant according to claim 2, characterized in that a second functional layer has a side which faces a third functional layer and is structured so that it constitutes an optimal substrate surface for the third functional layer, which ensures a firm positive or non-positive connection and a dispersion during the biomechanical loading of the third layer or the lowest possible shearing loads.

21. Implant according to claim 2, characterized in that a second functional layer has structures which increase the surface.

22. Implant according to claim 2, characterized in that a second functional layer has structures of which increase the surface that define indentations in the surface, (golf ball structure), undercut or drop-shaped indentations in the range from several pm to mm, or has web-like structures.

23. Implant according to claim 2, characterized in that a second functional layer has a side which faces a third functional layer and has a very slight microroughness in the region of a few μm.

24. Implant according to claim 4, characterized in that the ceramic material of the first and the second functional layers contains an oxide ceramic from the class of aluminum oxides or zirconium oxides or a non-oxide ceramic, for example materials based on Si₃N₄.

25. Implant according to claim 2, characterized in that a third functional layer contains three-dimensionally linked polymers.

26. Implant according to claim 2, characterized in that a third functional layer contains hydrogels.

27. Implant according to claim 26, characterized in that the third functional layer contains three-dimensionally linked polymers, wherein the internal structure of the polymers, in particular the internal structure of the hydrogels, is configured so that the cartilage formation is promoted under compressive mechanical load and with corresponding charging with chondrogenetic substances.

28. A method for restoring at least one joint surface in a human body comprising using the implant according to claim 1 for restoration of the at least one joint surfaces in the human body, the at least one joint surfaces being from the shoulder, hip, knee and/or foot regions of the human body.

29. A method according to claim 28, characterized in that the at least one joint surfaces includes, for local cartilage defects, partial joint replacements or full endoprostheses.

30. A method according to claim 28, characterized in that in articulations against natural cartilage surfaces.
